SEQUENCE LISTING (6 sequences)

SEQ ID NO: 1 - DNA, 18 bp, Artificial Sequence
  Synthetic oligonucleotide
ttcagccaaacgaccatc

SEQ ID NO: 2 - DNA, 18 bp, Artificial Sequence
  Synthetic oligonucleotide
caggttgcctctcactcg

SEQ ID NO: 3 - DNA, 18 bp, Artificial Sequence
  Synthetic oligonucleotide
aagttaggtgggcagctt

SEQ ID NO: 4 - DNA, 18 bp, Artificial Sequence
  Synthetic oligonucleotide
gggtgatcctcttctgct

SEQ ID NO: 5 - DNA, 18 bp, Artificial Sequence
  Synthetic oligonucleotide
tgccctgaggcactcttc

SEQ ID NO: 6 - DNA, 18 bp, Artificial Sequence
  Synthetic oligonucleotide
gtgccagggcagtgatct

(19) United States
(12) Patent Application Publication
(10) Pub. No.: US 2012/0295804 A1
(43) Pub. Date: Nov. 22, 2012

(54) Compositions and Methods for Diagnosis and Treatment of Breast Cancer

(75) Inventors: Pranela Rameshwar, Maplewood, NJ (US); Lillian F. Pliner, Short Hills, NJ (US); Margarette Bryan, Bloomfield, NJ (US)

(73) Assignee: University of Medicine and Dentistry of New Jersey, Somerset, NJ (US)

(21) Appl. No.: 13/473,938

(22) Filed: May 17, 2012

Related U.S. Application Data
(60) Provisional application No. 61/487,378, filed on May 18, 2011.

(51) Int. Cl. C12N 5/095; C12Q 1/18; C12Q 1/68; C40B 30/04; G01N 21/76; G01N 21/64

(52) U.S. Cl. 506/9; 435/371; 435/7.23; 435/6.12; 435/32

(57) ABSTRACT

The present invention is a population of breast cancer cells with preference for establishing dormancy in bone marrow. The breast cancer cells have characteristics of stem cells and express high levels of Oct4, designated Oct4^{hi}, but are also not dependent on stem cell gene status. The Oct4^{hi} cells exhibit functional gap junction intercellular communication with bone marrow stroma, indicating that these cells can establish dormancy and remain resistant to chemotherapy. Also provided by the present invention are a biomarker for metastatic breast cancer, a method for diagnosis and prognosis of breast cancer, and a method for identifying treatments that target dormant metastatic cells.

### INTRODUCTION

This application claims the benefit of U.S. Provisional Application No. 61/487,378, filed May 18, 2011, which is herein incorporated by reference in its entirety.

This invention was made with government support under Grant No. W81XWH-10-1-0413 awarded by the U.S. Department of Defense. The government has certain rights in the present invention.

### BACKGROUND OF THE INVENTION

The incidence of breast cancer remains relatively high although recent advancements in treatment modalities have improved overall survival. In the United States, breast cancer represents over one-fifth of all cancers (Kakarala, M. and M. S. Wicha. 2008. J. Clin. Oncol. 26:2813-2820). About 10% of hereditary breast cancer has been linked to highly penetrant mutations in BRCA1 and BRCA2 (Simon, M. S, and N. Petrucelli. 2009. Methods Mol. Biol. 471:487-500; Vega, A. et al. 2009. Gynecol. Oncol. 112:210-214). However, 90-95% of breast cancer is sporadic and cannot be attributed to any currently known germline mutations (Simon, M. S, and N. Petrucelli. 2009. Methods Mol. Biol. 471:487-500; Vega, A. et al. 2009. Gynecol. Oncol. 112:210-214). Environmental factors appear to play an important role in sporadic cases (Chia, K. S. 2008. Novartis Found. Symp. 293:143-150). In addition, stress and its associated hormones have been linked to cancer progression (Arranz, A. et al. 2010. Mol. Cancer. 9:261). Breast cancer patients with high body mass indices show poor clinical outcome regardless of hormone receptor/HER2 status of the primary cancer (Phipps, A. I. et al. 2011. Cancer Epidemiol. Biomarkers Prev. 20:454-463).

Breast cancer cells (BCCs) have a predilection to metastasize to the bone marrow, brain, liver and lung (Cocoran, K. E. et al. 2008. PLoS One 3:e2563; Kakarala, M. and M. S. Wicha. 2008. J. Clin. Oncol. 26:2813-2820). In bone marrow, BCCs can form gap junctional intercellular communication (GJIC) with stroma, close to the endosteum (Cocoran, K. E. et al. 2008. PLoS One 3:e2563). These findings are consistent with earlier studies that reported on slower growth rates of BCCs close to the endosteum of mice (Rao, G. et al. 2004. Cancer Res. 64:2874-2881). Loss of connexin 43, a gap junction protein, is linked to malignancy in cancers, breast cancer included (Bodenstine, T. M. et al. 2010. Cancer Res. 70:10002-10011; King, T. J. et al. 2002. Mol. Carcinog. 35:29-41). A key role for GJIC in the quiescence of BCCs within the stromal compartment of bone marrow has been recently reported, with activity attributed to movement of microRNAs from stroma to BCCs for reduced cell cycle activity (Lim, P. K. et al. 2011. Cancer Res. 71:1550-1560). Despite the involvement of GJIC as a facilitator of BCC dormancy, connexins cannot be directly targeted since hematopoietic activity depends on GJIC among bone marrow stromal cells (Milson, M. D. and A. Trump. 2011. Nat. Immunol. 12:377-379).

Octamer-binding transcription factor 3/4 (Oct4) is a member of the POU DNA-binding domain family and a stem cell marker (Liedtke, S. et al. 2008. Biol. Chem. 389:845-850). There are three Oct4 mRNAs: Oct4A, Oct4B and Oct4B1 (Wang, X. and J. Dai. 2010. Stem Cells 28:885-893). The Oct4B transcript can use alternate translational start sites to generate three protein isoforms (Zhang, W. et al. 2010. Biochem. Biophys. Res. Commun. 394:750-754). Although the role of Oct4 in self-renewal of normal adult stem cells remains debatable, Oct4A seems to be relevant for maintaining pluripotency of embryonic stem cells (Wang, X. and J. Dai. 2010. Stem Cells 28:885-893). Oct4 is over-expressed in many tumor types, including breast cancer (Guzman-Ramirez, N. et al. 2009. Prostate 69:1683-1693; Hu, T. et al. 2008. Cancer Res. 68:6533-6540; Suva, M. L. et al. 2009. Cancer Res. 69:1776-1781; Zhang, S. et al. 2008. Cancer Res. 68:4311-4320). Oct4 mediates chemotherapy drug resistance in hepatocellular carcinoma, lung and prostate cancer, and maintains self-renewal of lung cancer stem cells (Wang, X. Q. et al. 2010. Hepatology 52:528-539; Chen, Y. C. et al. 2008. PLoS One 3:e2637). Furthermore, Oct4 expression maintains cell survival through the Oct4/Tcl1/Akt1 pathway in MCF7 BCCs (Hu, T. et al. 2008. Cancer Res. 68:6533-6540).

There have been several reports on the identification of breast cancer stem cells via surface marker expression, including CD44⁺/CD24⁻/lin⁻ or ALDH1⁺, or via efflux of Hoechst dye with the side population (Al-Hajj, M. et al. 2003. PNAS USA 100:3983-3988; Ginestier, C. et al. 2007. Cell Stem Cell 1:555-567; Liu, S, and M. S. Wicha. 2010. J. Clin. Oncol. 28:4006-4012). Researchers have found that certain breast cancer stem cell types, specifically CD44⁺CD24^{-/low} cells that express Oct4, are potential targets for chemotherapy (Eriksson et al. 2007. Mol. Ther. 15:2088-2093) or drug treatments for breast cancer.

It is evident that not all BCCs are functionally equal and perhaps a particular subset is responsible for the formation of GJIC with bone marrow stroma (Lim, P. K. et al. 2011. Cancer Res. 71:1550-1560). It has now been found that a subset of BCCs with self-renewal and tumor-initiating properties shows preference for GJIC with bone marrow stroma. The identity of BCC subset with preference for dormancy in bone marrow, and perhaps in other organs, provides important information about the mechanism by which BCCs adapt dormancy and also provides an understanding of how the process could potentially be reversed to prevent tertiary metastasis (Cocoran, K. E. et al. 2008. PLoS One 3:e2563; Lim, P. K. et al. 2011. Cancer Res. 71:1550-1560).

### SUMMARY OF THE INVENTION

The present invention is a breast cancer biomarker which comprises a breast cancer cell with a phenotype OCt4^{hi}/CD44^{hi/med}/CD24^{-/+}. In the present invention it has been shown that cells with the OCt4^{hi}/CD44^{hi/med}/CD24^{-/+} phenotype represent dormant, metastatic breast cancer cells that can exist in bone marrow. As a result, the breast cancer cells of the present invention establish gap junctional intercellular communication with bone marrow stroma.

The present invention is also a method for developing a breast cancer prognosis for a patient which comprises detecting the presence of the biomarker cells of the present invention in a patient sample wherein the presence of said biomarker is indicative of a the presence of dormant metastatic breast cancer cells in the patient and a poor prognosis for the patient. Additionally, the present invention is a method of diagnosing metastatic breast cancer which comprises detecting the presence of the biomarker of the present invention in a patient sample, wherein the presence of the biomarker indicates that the patient has metastatic breast cancer.

Yet another object of the present invention is a method for identifying chemotherapeutic agents that can kill dormant metastatic breast cancer cells in bone marrow which comprises that steps of contacting a breast cancer cell with a phenotype Oct4^{hi}/CD44^{hi/med}/CD24^{-/+} in vitro with an agent; and determining whether the agent is capable of killing said breast cancer cell, wherein death of said breast cancer cell is indicative of the ability of the agent to kill dormant metastatic breast cancer cells in bone marrow.

### BRIEF DESCRIPTION OF THE DRAWINGS

FIG. 1 depicts results of experiments to determine baseline Oct4 expression levels in BCC lines and primary cells. Real-time PCR was performed with total RNA from MDA-MB-231 and T47D. The Ct values were plotted on the y-axis (n=8).

FIG. 2 depicts results of experiments to determine baseline Oct4 expression levels in BCC lines and primary cells. Peripheral blood mononuclear cells from patients with breast cancer were double-labeled with fluorochrome conjugated anti-cytokeratin (PE) and anti-Oct4 (FITC). The analyses were performed with all cells, but the Oct4 expressers were analyzed by setting the threshold on PE emission. Shown are representative of five analyses: Patient 1 (left panel); Patient 2 (middle panel); Patient 4 (right panel).

FIG. 3 depicts results of experiments to determine relative proliferation rates of BCC subsets. Freshly sorted BCCs were assessed for doubling time using CyQUANT Cell Proliferation Assay Kit. The results are presented as mean±SD, n=4. **p<0.05 vs. other subsets; *p<0.05 vs. unsorted and Oct4⁻ subset.

FIG. 4 depicts results where unsorted and sorted BCC subsets were studied for cell cycle phase by propidium iodide incorporation and then analyzed by flow cytometry. The results were similar for both cell lines. Shown are the densitometric analyses of western blots from MDA-MB-231 cells. Bands were normalized to β-actin and then presented as normalized densities±SD, n=3.

FIG. 5 depicts results where unsorted and sorted BCC subsets were studied for cell cycle phase by propidium iodide incorporation and then analyzed by flow cytometry. The results were similar for both cell lines. Shown are the data for MDA-MB-231 cells.

FIG. 6 depicts further results of proliferation rates of BCCs. Oct4⁺ and Oct4⁻ BCCs, monitored by time-lapse imaging, were assessed for cell cycle time, which was measured as the time between anaphases of the parental and daughter cells. Data indicate the mean cell cycle time of 50 cells.

FIG. 7 depicts results of experiments of dye retention in BCC subsets. Oct4^{hi} BCCs were labeled with Hoechst 33342 (5 μg/mL), in the presence or absence of 400 μM verapamil. Propidium iodide-negative cells were gated based on GFP intensity. The cells were analyzed on Hoechst Blue and Hoechst Red filters. Side population cells that were Hoechst negative (circled) were compared in the two experimental conditions. The figure represents five different experiments, with MDA-MB-231 and T47D.

FIG. 8 depicts results of experiments of dye retention in BCC subsets. Oct4^{med} BCCs were labeled with Hoechst 33342 (5 μg/mL), in the presence or absence of 400 μM verapamil. Propidium iodide-negative cells were gated based on GFP intensity. The cells were analyzed on Hoechst Blue and Hoechst Red filters. Side population cells that were Hoechst negative (circled) were compared in the two experimental conditions. The figure represents five different experiments, with MDA-MB-231 and T47D.

FIG. 9 depicts results of experiments of dye retention in BCC subsets. Oct4^{low} BCCs were labeled with Hoechst 33342 (5 μg/mL), in the presence or absence of 400 μM verapamil. Propidium iodide-negative cells were gated based on GFP intensity. The cells were analyzed on Hoechst Blue and Hoechst Red filters. Side population cells that were Hoechst negative (circled) were compared in the two experimental conditions. The figure represents five different experiments, with MDA-MB-231 and T47D.

FIG. 10 depicts densitometric analyses of western blots that were performed for ABCG2 with whole cell extracts from unseparated and different subsets of BCCs.

FIG. 11 depicts the results of experiments to examine stem cell gene expression in BCC subsets. Oct4⁻ and Oct4^{hi} subsets from MDA-MB-231 cell line were analyzed for stem cell genes using total RNA and Tagman Stem Cell Pluripotency Array. The output values were normalized to internal control and the 2^{ΔΔCt} values were calculated (Oct4^{hi}/Oct4⁻).

FIG. 12 depicts the results of experiments to examine stem cell gene expression in BCC subsets. Oct4⁻ and Oct4^{hi} subsets from T47D cell line were analyzed for stem cell genes using total RNA and Taqman Stem Cell Pluripotency Array. The output values were normalized to internal control and the 2^{ΔΔCt} values were calculated (Oct4^{hi}/Oct4⁻).

FIG. 13 shows results of stem cell gene expression profiles in BCC subsets (Oct4⁻ and Oct4^{hi}) using total RNA and Taqman Stem Cell Pluripotency Array. The output values were normalized to internal control and the 2^{ΔΔCt} values were calculated (Oct4^{hi}/Oct4⁻). The fold changes of genes showing >1.5 fold differences in expression, were analyzed with Ingenuity Pathway Analysis.

FIG. 14 shows results of stem cell gene expression profiles in BCC subsets (Oct4⁻ and Oct4^{hi}) using total RNA and Taqman Stem Cell Pluripotency Array. The output values were normalized to internal control and the 2^{ΔΔCt} values were calculated (Oct4^{hi}/Oct4⁻). The fold changes of genes showing <0.9 fold differences in expression, were analyzed with Ingenuity Pathway Analysis.

FIG. 15 shows results of experiments of stem gene expression in BCCs. Western blots were performed for stem cell-associated proteins using nuclear extracts from MDA-MB-231 and T47D, except for Notch-1, which was analyzed with cytoplasmic extracts. Acetyl-histone H3 served as nuclear control and ribosomal protein L28 served as cytoplasmic control. Bands from western blots were analyzed for densities and then normalized with housekeeping genes. The results are presented as normalized densities±SD, n=3.

FIG. 16 depicts results of experiments in vivo examining tumor growth with different numbers of Oct4^{hi} BCCs in nude BALB/c. BC subsets (200 cells) were injected in the dorsal flank of nude BALB/c. Cells from Oct4^{hi} tumors were serially passaged up to four times. The results are presented as time (days) to achieve tumor volume of 0.5 cm³, n=5; mean with upper line=75th percentile and lower line=25^{th} percentile.

FIG. 17 depicts results of experiments in vivo examining tumor growth with different numbers of Oct4^{hi} BCCs in nude BALB/c. Oct4^{hi} BCCs and unsorted BCCs were compared for carboplatin sensitivity, which was injected via intraperitoneal route when the tumors were −0.5 cm³. *p<0.05 vs. unsorted BCC.

FIG. 18 depicts results of experiments examining GJIC between Oct4^{hi} cells and bone marrow stroma. Western blots for connexin proteins were performed with plasma membrane extracts from subsets of MDA-MB-231 and T47D. Bands from western blots were analyzed for densities and then normalized with the housekeeping genes. The results are presented as normalized densities±SD, n=3.

FIG. 19 depicts results of experiments examining GJIC between Oct4^{hi} cells and bone marrow stroma GJIC frequency was calculated based on the total number of BCC showing dye transfer into stroma in co-cultures. The frequency was calculated as the percent dye transfer from total number of seeded BCCs. Data is presented as mean frequency±SD, n=4.

FIG. 20 presents the working hierarchy of BCCs that was created based on functional and phenotypic studies. Hierarchy assimilates Oct4, CD44, and CD24 expression status with consideration for GJIC formation ability and self-renewal ability.

### DETAILED DESCRIPTION OF THE INVENTION

The identity of tumor-initiating cells in breast cancer has been examined, however, there is no clear consensus on the exact phenotype of this population. The expression of stem cell markers such as Oct4, Sox2, and Nanog has been linked to breast cancer (Bourguignon, L. Y. et al. 2008. J. Biol. Chem. 283:17635-17651; Hu, T. et al. 2008. Cancer Res. 68:6533-6540; Lengerke, C. et al. 2011. BMC Cancer 11:42). The present invention is focused on Oct4-expressing cells, based on the role of this gene as a regulator of other stem cell genes (Yang, J. et al. 2010. PLoS One 5:e10766), as well as its detection in adult and embryonic stem cells (Greco, D. J. et al. 2007. Stem Cells 25:3143-3154; Johansson, H. and S. Simonsson. 2010. Aging 2:815-822). Oct4 exists as multiple isoforms, e.g., Oct4A, Oct4B, and Oct4B1 (Asadi, M. H. et al. 2010. Int. J. Cancer 128:2645-2652). In the present invention, the status of cells as being either Oct4-expressing (Oct4⁺) or lacking expression of Oct4 (Oct4⁻) was not the only basis for identifying cells. Instead, Oct4⁺ or Oct4⁻ cells have been subdivided in the instant invention, depending on the level of Oct4 gene expression. To identify subsets of BCCs, BCCs were stably transfected with pEGFP1-Oct3/4 and different BCC subsets were then isolated based on the intensity of GFP emission, since GFP emission was shown to correlate with Oct4 protein levels. Studies with the stably transfected BCCs indicated that the subset with the highest Oct4 expression consisted of breast cancer-initiating cells that expressed certain stem cell genes.

Thus, the present invention is a population of BCCs with preference for establishing dormancy in bone marrow. This subset of BSCs shows self-renewal, divides asymmetrically, has a long doubling time, exhibits tumor-initiating properties, and expresses high levels of Oct4 and other stem cell-associated genes. Moreover, the breast cancer stem cells identified as being high Oct4 expressing cells, designated Oct4^{hi}, have been shown to be independent of CD44/CD24 status, which has been reported by others to be a critical feature of breast cancer stem cells. Also of importance to the instant invention is the finding that the breast cancer stem cells expressing high levels of Oct4 exhibit functional gap junction intercellular communication (GJIC) with bone marrow stroma, indicating that these cells can establish dormancy and remain resistant to chemotherapy. Thus, targeting these particular BCCs provides a new tool for therapy of breast cancer. Finally, a hierarchy of BCC subsets has also been identified, which can be used as a basis to identify combinations of markers to demarcate cancer cell subsets for diagnosis and prognosis, as well as to develop novel treatments and further the understanding of breast cancer dormancy and resurgence. These discoveries have led to the compositions and methods of the present invention. Hence, the present invention provides a novel BCC subset that can be exploited in the diagnosis and treatment of breast cancer.

A role of miRNA and GJIC in BCC quiescence within the stromal compartment of bone marrow was recently reported (Lim, P. K. et al. 2011. Cancer Res. 71:1550-1560). Based on this finding, experiments were performed to identify of BCC subset or subsets that are responsible for cancer cell dormancy. Initial experiments focused on Oct4-expressing BCCs because of its involvement in pluripotency and tumorigenesis. Relevant Oct4 isoforms expressed in breast cancer were identified by screening a panel of eight BCC lines, by western blots with an antibody that detects all isoforms of Oct4. The eight BCC lines studied included MDA-MB-231, MDA-MB-468, MDA-MB-453, T47D, MCF-7, HCC1954, HCC1143, and BT549. The results showed light bands at the predicted size for Oct4A (45 kDa) and undetectable bands at the regions expected for Oct4B proteins in all cell lines. Positive controls, using extracts from mesenchymal stem cells, showed predictable bands for Oct4B at 18, 21 and 29 kDa. These data provided evidence of the role of Oct4A as a mediator of pluripotency and the inability of Oct4B to sustain self-renewal (Wang, X. and J. Dai. 2010. Stem Cells 28:885-893).

BBC lines MDA-MB-231 and T47D were selected for subsequent studies due to their established differences in hormone receptor expression. RT-PCR with primers specific for Oct4A and Oct4B showed bands at the predicted size (350 bp) for both transcripts, although detectable bands for Oct4B required 40 cycles. The primers used are shown below in Table 1. Real-time RT-PCR confirmed lower Ct values for Oct4A indicating higher levels of its mRNA.

TABLE 1
Primer Sequences

| | | SEQ |
|---|---|---|
| | | ID |
| Primer | Sequence(5′ → 3′) | NO: |
| Oct4A-Forward | TTC AGC CAA ACG ACC ATC | 1 |
| Oct4A-Reverse | CAG GTT GCC TCT CAC TCG | 2 |
| Oct4B-Forward | AAG TTA GGT GGG CAG CTT | 3 |
| Oct4B-Reverse | GGG TGA TCC TCT TCT GCT | 4 |
| β-actin-Forward | TGC CCT GAG GCA CTC TTC | 5 |
| β-actin-Reverse | GTG CCA GGG CAG TGA TCT | 6 |

Due to the relatively low expression of Oct4, experiments were performed to determine if the low expression could be explained by low frequency of Oct4⁺ BCCs in the cell lines. Immunocytochemistry was performed in MDA-MB-231 and T47D cells in culture with the same anti-Oct4 antibody used in the western blots. Cells that stained bright green were designated as Oct4^{+/hi} BCCs and were found in the following frequencies: 1.0%±0.37 for MDA-MB-231 and 0.84±0.17 for T47D (mean±SD, n=5). Thus, the expression of Oct4 was low in these cell lines, a finding that is consistent with low frequency of other stem cells. Although the levels of expression were low in the cell lines, the Oct4⁺ cells did cluster.

Oct4 expression was next studied in primary human breast cancer tissue samples and compared with normal tissues surrounding the malignant regions. The demographics of patients that served as sources for tissue are described in Table 2. Western blot analyses indicated low levels of Oct4 protein in the patient tumor samples and undetectable expression in the control, non-malignant tissue. Hematoxylin-eosin staining showed no evidence of tissue necrosis. Parallel staining for Oct4 protein indicated intense labeling for the highly malignant tissue with moderately to poorly differentiated areas. Similar staining with the surrounding normal tissue showed light to negative staining.

TABLE 2
Demographics of Patients that Served as Breast Cancer Tissue Sources

| Sub- | | | | | | |
|---|---|---|---|---|---|---|
| jects | | | Tumor | | | |
| (S) | Stage | Grade | Size | Histology | ER/PR | HER2 |
| S1* | IIIA | Intermediate | T2 | Infiltrating | Negative | Negative |
| | | | | ductal | | |
| | | | | carcinoma | | |
| S2 | IIB | Intermediate | T3 | Invasive | Unknown | Unknown |
| | | | | ductal | | |
| | | | | carcinoma | | |
| S3 | IIIC | Intermediate | T3 | Infiltrating | Positive | Positive |
| | | | | ductal | | |
| | | | | carcinoma | | |
| S4 | IIIA | High | T2 | Infiltrating | Unknown | Unknown |
| | | | | ductal | | |
| | | | | carcinoma | | |
| S5 | IIA | High | T2 | Invasive | Negative | Negative |
| | | | | ductal | | |
| | | | | carcinoma | | |
| S6 | IIIC | High | T2 | Infiltrating | Unknown | Unknown |
| | | | | ductal | | |
| | | | | carcinoma | | |

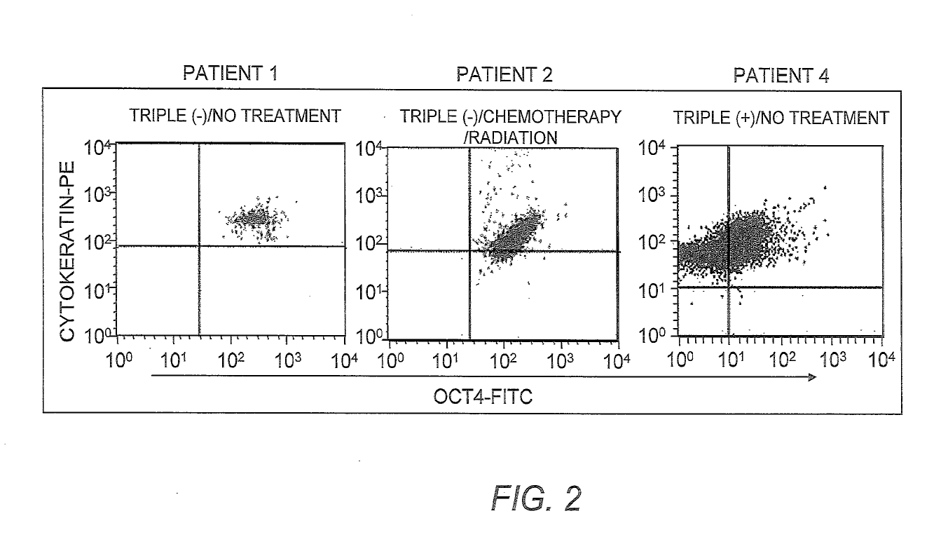

Experiments were then performed to determine if Oct4⁺ BCCs can be identified in the blood of patients presenting with different stages of breast cancer, including and excluding treatment (Table 3). Results of flow cytometry on blood samples from three representative patients are shown in FIG. 2. Patients 1 and 2 had tumors that were hormone receptor negative (−), while Patient 4 was hormone receptor positive (+). Circulating BCCs positive for cytokeratin that co-expressed Oct4 were observed in all patients (FIG. 2). Patient 1 was examined before treatment and presented with 36 lymph nodes negative for tumor cells, despite a 12 cm tumor at the primary site. Patient 2 was subjected to six cycles of chemotherapy and six cycles of radiation. Patient 4 was analyzed before treatment and showed both Oct4⁺ and Oct4⁻ cytokeratin⁺ cells. Patient 6 (hormone receptor+) was treated and also showed results similar to Patient 4 (not shown). The wide range of Oct4 fluorescence intensities suggested heterogeneity with respect to Oct4 levels in circulating BCCs. These data also indicated that primary cancer cells could enter blood and not lymph nodes of patients, particularly in view of the results of Patient 1 where there was a large tumor but 36 negative lymph nodes, as well as the results in a patient that had completed six cycles of chemotherapy and radiation but still showed detectable levels in blood (Patient 2).

TABLE 3
Data for Patients that Were Sources of Peripheral Blood Samples

| | | | | | Documented | | |
|---|---|---|---|---|---|---|---|
| | | ER/PR | HER2 | | distant | | |
| Patients | Stage | status | status | Treatment | metastasis? | Obese | Other |
| P1 | IIIB | Neg | Neg | None | No | No | T4 |
| | | | | | | | tumor. |
| | | | | | | | Patient |
| | | | | | | | was not |
| | | | | | | | treated |
| P2 | III | Neg | Neg | Chemotherapy/ | No | No | |
| | | | | Radiation | | | |
| P3 | IIIA | Pos | Unknown | Chemo, then | No | No | Tumor |
| | | | | surgery | | | and |
| | | | | | | | nodes |
| | | | | | | | present |
| P4 | IIA | Pos | Pos | None | Yes | Yes | |
| P5 | N/A | Pos | N/A | None | No | Yes | |
| P6 | III | Neg | Neg | Surgery, | No | No | |
| | | | | then chemo | | | |
| P7 | III | Pos | Neg | | No | Yes | |

The low frequencies of Oct4⁺ BCCs within the eight cell lines examined (<1%), as well as their resistance to chemotherapy in patients indicated that the level of Oct4 expression may be correlated with the maturity of the BCC. Therefore, different BCC subsets were selected based on Oct4 expression. To do this, MDA-MB-231 and T47D were stably transfected with an Oct4 reporter vector, pEGFP1-Oct3/4 (Gerrard, L. et al. 2005. Stem Cells 23:124-133). The transfected cells were sorted into three subsets based on GFP expression levels. The top 5% of GFP expressers were designated Oct4^{hi} and the lowest 5%, Oct4⁻. The population between the extremes were designated Oct4^{med}. GFP intensities were validated for correlation with Oct4 protein by intracellular flow cytometry of pEGFP1-Oct3/4 transfectants. The Oct4⁻ population was similar to isotype control. In contrast, the mean fluorescence intensity (MFI) of cells labeled with APC-anti-Oct4 correlated with GFP intensities. The MFI of Oct4^{hi} was approximately 8 fold higher than Oct4^{med}, indicating that GFP intensity was proportional to Oct4 protein levels. The Oct4⁻ population was not due to the loss of pEGFP1-Oct3/4 since treatment of the stable transfectants with a G9a histone methyltransferase inhibitor, BIX01294 (Shi, Y. et al. 2008. Cell Stem Cell 3:568-574), induced GFP expression in which 92.5%±4.4% of the Oct4⁻ BCCs reverted to high GFP expression.

Experiments were then performed to quantify the frequency of tumorsphere formation in BCC subsets. In order to accurately quantify the frequency of tumorsphere formation in serial passages, the stability of Oct4^{hi} cells in culture was first examined. After two weeks in culture, the sorted Oct4^{hi} BCCs emerged as two distinct populations with regard to GFP intensities. The percentage of Oct4^{hi} cells was 36.5±5.9 (mean±SD, n=15), suggesting that the Oct4^{hi} cells differentiated rapidly in culture. This could not be explained by contaminants from Oct4⁻ cells during sorting since all cells were Oct4^{hi} immediately after sorting. Furthermore, only singlet was sorted, indicating that the Oct4^{hi} cells could not be explained by doublets of two low GFP cells. Based on the instability of cultures to maintain Oct4^{hi} BCCs, subsequent experiments were performed with freshly sorted cells.

Three sorted BCC subsets were then compared for tumorsphere formation. Oct4^{hi} cells, when plated at 1 cell/well, formed a tumorsphere in low attachment 96-well plates, whereas Oct4^{med} and Oct4⁻ cells did not form tumorspheres. Oct4^{med} cells showed small clusters of less than 20 cells, which were then designated as negative tumorsphere. Single-cell derived tumorspheres from Oct4^{hi} BCCs (MDA-MB-231 and T47D) were tested for serial passaging in low attachment plates four times. The frequencies of tumorsphere formation from the parental unsorted cells, Oct4^{med}, and Oct4^{hi} BCCs, showed similar results for both MDA-MB-231 and T47D. Oct4^{hi} BCCs showed greater than 96% efficiency in tumorsphere formation at each passage. The tumorsphere sizes in each passage were comparable, suggesting similar cellular maturity. The small tumorspheres from Oct4^{med} BCCs could not be serially passed. Of note, cells at the periphery of the tumorspheres showed a decrease in GFP signal, suggesting differentiation within the tumorsphere. It was concluded that since PO Oct4⁻ BCCs did not form tumorspheres, these were differentiated as compared to the primitive status of Oct4^{hi} cells.

Evidence of self-renewal in noble agar was investigated with the three BCC subsets. The cultures in noble agar were followed and the images captured at the time when one Oct4^{hi} BCC formed two cells. Comparable GFP intensities were observed for the two cells, suggesting self-renewal. The half-life of GFP is about 20 hours. Since the GFP intensities of the parent and daughter cells were similar, the results indicated that continued expression of Oct4 must be replacing the degraded GFP. The progeny of Oct4^{med} showed reduced GFP intensity. The Oct4⁻ BCCs divided into two cells, with undetectable GFP.

The two-cells from the Oct4^{hi} cultures were expanded for two weeks in suspension cultures. The cells in the suspension cultures were re-plated in noble agar and the results showed colonies of cells with varied GFP intensities and different sizes, indicating that the original cloned cell generated heterogeneous populations of BCCs. These data indicated that the original cell was tumor-initiating with self-renewal properties. Oct4^{med} and Oct4⁻ cells did not expand into cells that can form colonies in noble agar.

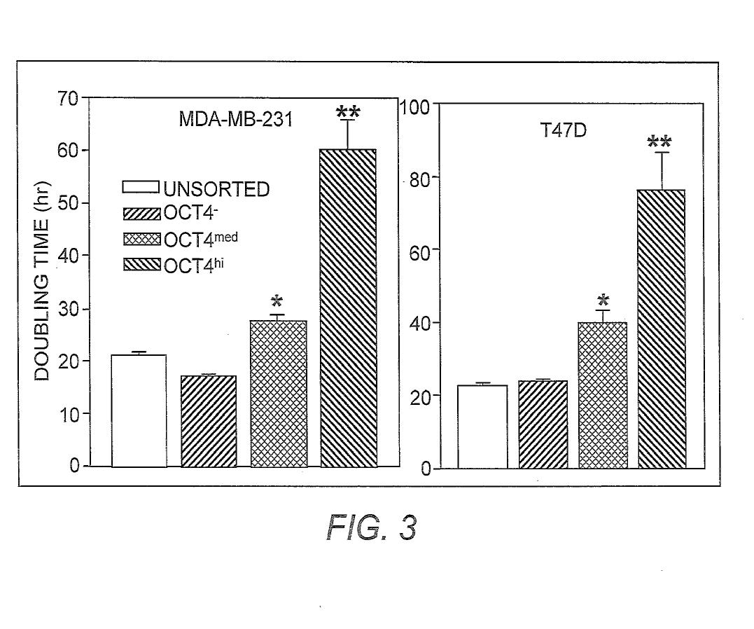

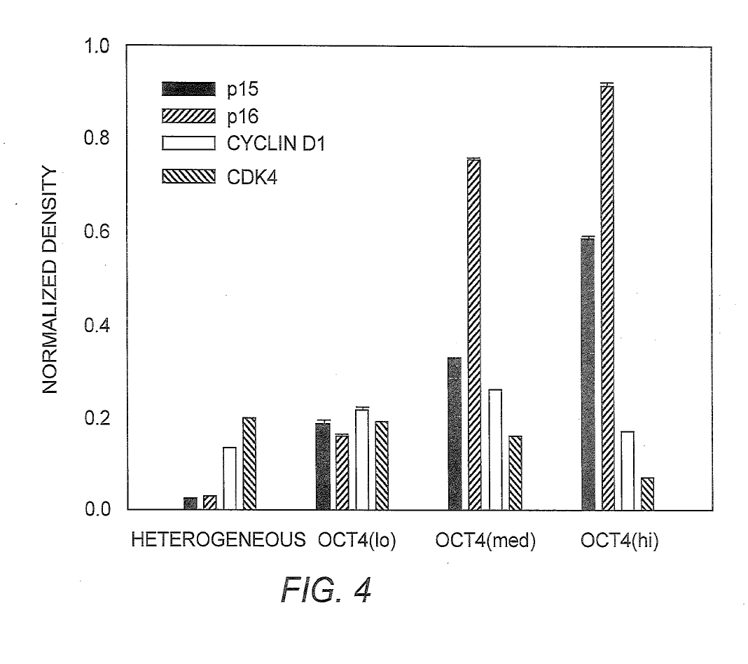

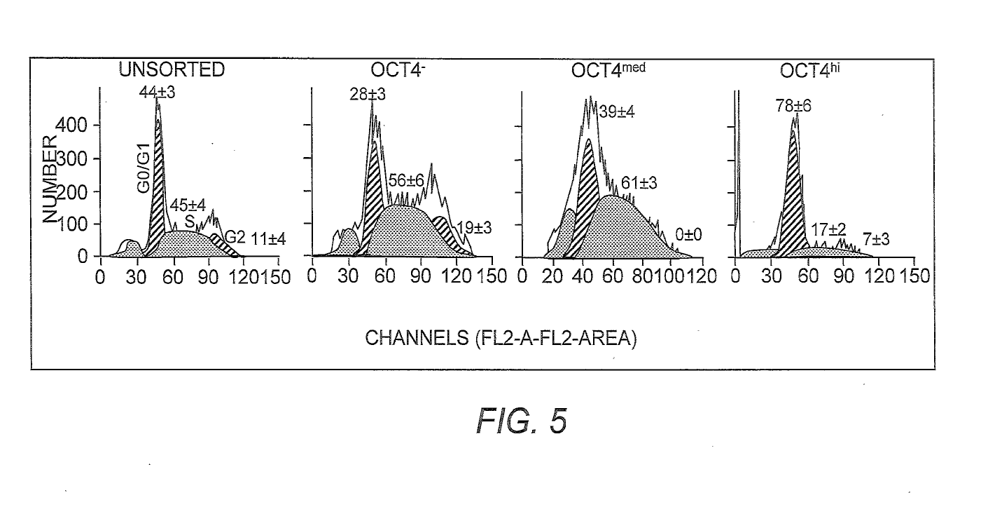

The cell cycle status of BCC subsets was then examined; specifically, the cell cycle phase of Oct4^{hi}, Oct4^{med} and Oct4⁻ BCCs was compared. Doubling times in proliferation assays were determined. Oct4^{hi} BCCs showed approximately 3-fold greater doubling time than unsorted, Oct4^{med} and Oct4⁻ BCCs (FIG. 3). Next, the findings were validated by western blots for cell cycle proteins. There were increases in G₁-linked p15 and p16 with nuclear extracts from Oct4^{hi} cells, but reduced bands for Oct4^{med} and Oct4⁻ and decreases in G₁/S transition proteins (Cyclin D1 and Cdk 4) in the Oct4^{hi} subset (FIG. 4). Next, cell cycle analyses were performed using propidium iodide staining to distinguish G₀/G₁ (quiescence) from S/G₂/M (rapid cycling) phase. The results indicated that greater than 75% of Oct4^{hi} cells were in G₀/G₁ phase (FIG. 5).

Relative proliferation rates of BCC subsets were determined, specifically rates of proliferation for Oct4^{hi} and Oct4⁻. Bright-field and fluorescence images of cell divisions of representative Oct4⁺ and Oct4⁻ cells were obtained by video time-lapse microscopy at different time points. The first cell division for Oct4⁺ cells was 7 hours after the start of time-lapse imaging, and the cell cycle times of the daughter cells were markedly different. One daughter divided at 38 hours, whereas the other daughter did not divide until 64 hours. The cell division at 38 hours resulted in three cells, and one of the cells died. In contrast, for the Oct4⁻ cell, the cell cycle rates of the daughters and granddaughters were similar. The first cell division occurred 2 hours after the start of time-lapse imaging, and the second and third divisions of all the progeny occurred by 30 hours and 59 hours, respectively.

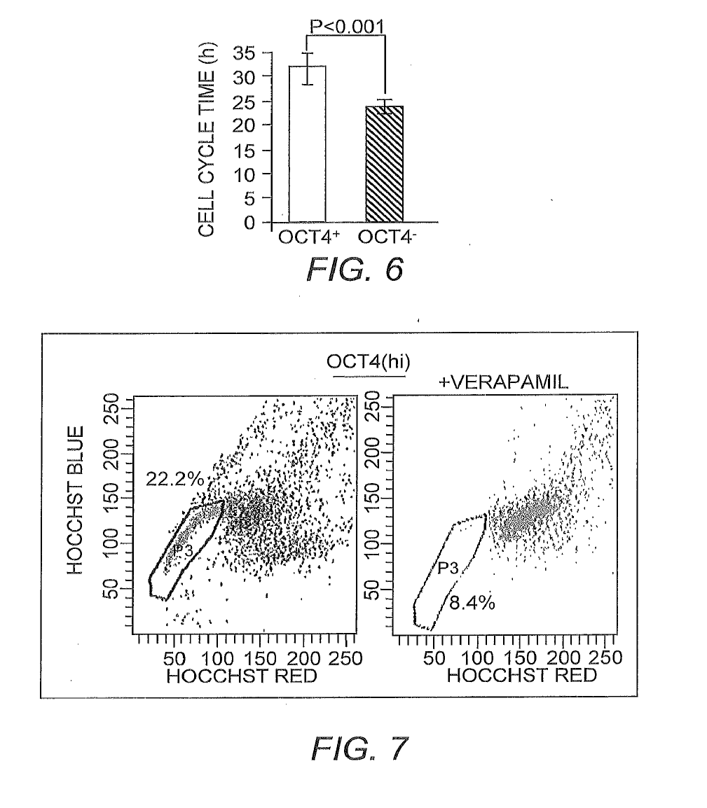

The production of mixed colonies from single Oct4^{hi} cells, and the images from the time lapse studies, indicated that Oct4^{hi} cells may be asymmetrically dividing. Recent evidence has suggested that when stem cells asymmetrically divide, they give rise to daughters with differing proliferative rates (Cicalese, A. et al. 2009. Cell 138:1083-1095; Costa, M. R. et al. 2011. Development 138:1057-1068). To test whether BCC subsets gave rise to daughters with differing proliferative rates, MDA-MB-231 cell divisions were monitored and the daughters tracked by time-lapse video microscopy. Due to the qualitative nature of visualizing GFP by immunofluorescence, cells were classified as Oct4⁻ and Oct4⁺, rather than the Oct4⁻, Oct4^{med} and Oct4^{hi}. Cell cycle times of individual Oct4⁻ and Oct4⁺ cells were monitored. Unlike the population doubling times calculated from cell proliferation (FIG. 3), the individual cell cycle times were calculated based on real-time analyses. As expected (FIG. 3), the Oct4⁺ cells had a significantly longer cell cycle time as compared to Oct4⁻ cells (mean time=32 hours and 24 hours, respectively, p<0.001) (FIG. 6). The cell cycle time of individual Oct4⁺ cells varied from 24 to >68 hours, whereas Oct4⁻ cell cycle times varied from 20 to 29 hours, suggesting that Oct4⁺ cells give rise to daughters of differing proliferative rates. To test this directly, live imaging of multiple cell divisions was performed over 68 hours, the daughter generation times of Oct4⁺ and Oct4⁻ cells were monitored, and lineage trees of the progeny were generated. The proliferative rates of progeny from Oct4⁻ cells were very similar to each other, but there were marked differences in proliferative rates among daughters of Oct4⁺ cells. Symmetric cell divisions were then defined as a difference in daughter cell cycle length of more than 8 hours, or approximately one-third the doubling time of the cell line. Over 47% of Oct4⁺ cells asymmetrically divided whereas none of the Oct4⁻ cells divided asymmetrically. Of note, none of the several thousand Oct4⁻ cells monitored by time-lapse microscopy became Oct4⁺ cells, lending further support to a hierarchical organization of BCC subsets under normal culture conditions.

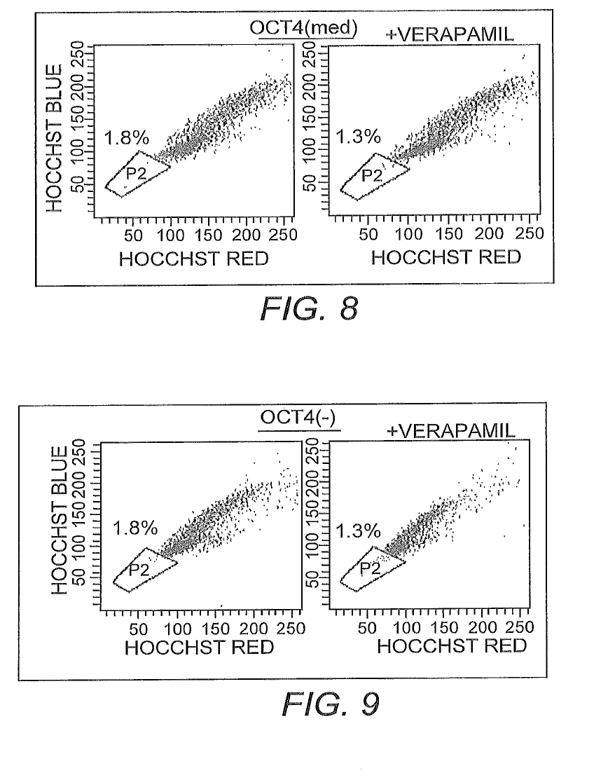

Experiments were then performed to determine if drug resistance genes played any role in the differentiation of BCC subsets. Since Oct4^{hi} BCCs showed an immature phenotype, this subset was tested for its ability to exclude Hoechst dye, a characteristic commonly reported for stem cells. Oct4^{hi} cells were incubated in the presence or absence of verapamil, which inhibits multi-drug resistance transporters, and then dye exclusion was tested in four different experiments. Oct4^{hi}, Oct4^{med} and Oct4⁻ cells were tested. The Oct4^{hi} population showed a 3-fold difference in dye retention with verapamil (FIG. 7). Only few Oct4^{med} (FIG. 8) and Oct4⁻ cells (FIG. 9) excluded the dye.

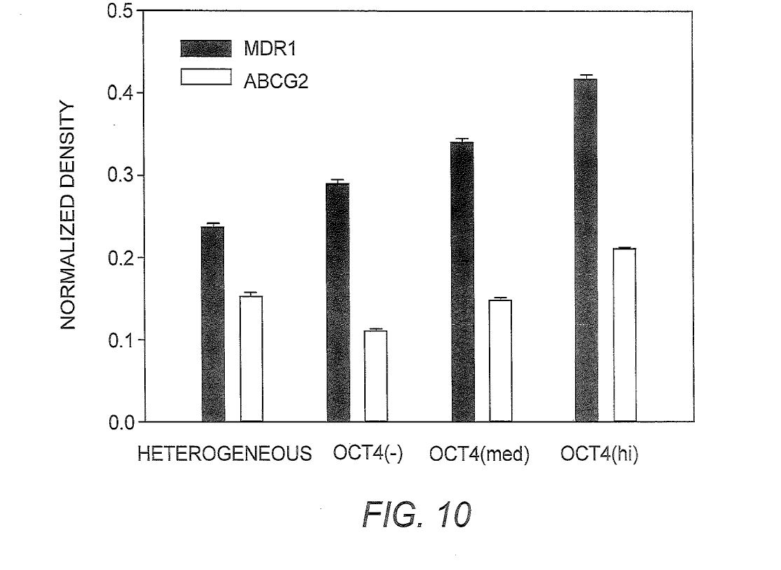

It was then determined if different expressions of ABCG2 and MDR1 drug resistance genes could explain the results of the Hoechst dye exclusion experiments (FIGS. 6 through 8). Western blots for MDR1 and ABCG2 showed detectable bands with extracts from Oct4^{hi} cells and light to undetectable bands for unseparated, Oct4^{med} and Oct4⁻ BCCs (FIG. 10). Flow cytometry for membrane-bound MDR1 indicated low mean fluorescence intensities (MFI) for Oct4⁻ and heterogeneous BCCs as compared to Oct4^{med}, which indicated a small subset with higher expression of MDR1. In contrast, Oct4^{hi} cells showed one peak, indicating homogeneity for MDR1. The MFI for Oct4^{hi} cells were increased by 10-fold from Oct4^{med}. ABCG2 expression indicated two subsets in the heterogeneous and Oct4^{hi} cells. The MFIs of Oct4^{med} and Oct4⁻ BCCs were reduced as compared to Oct4^{hi} cells.

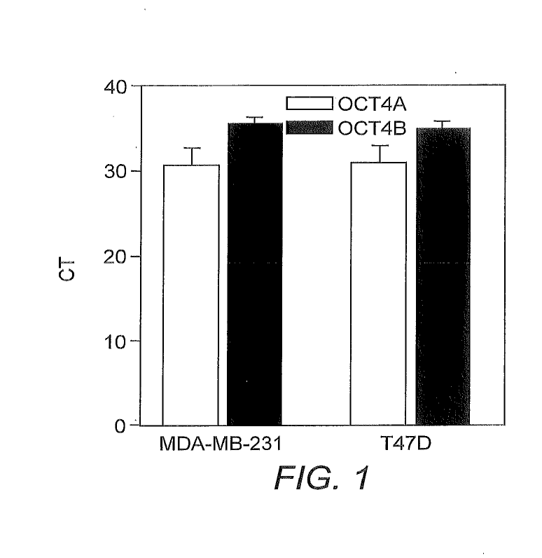

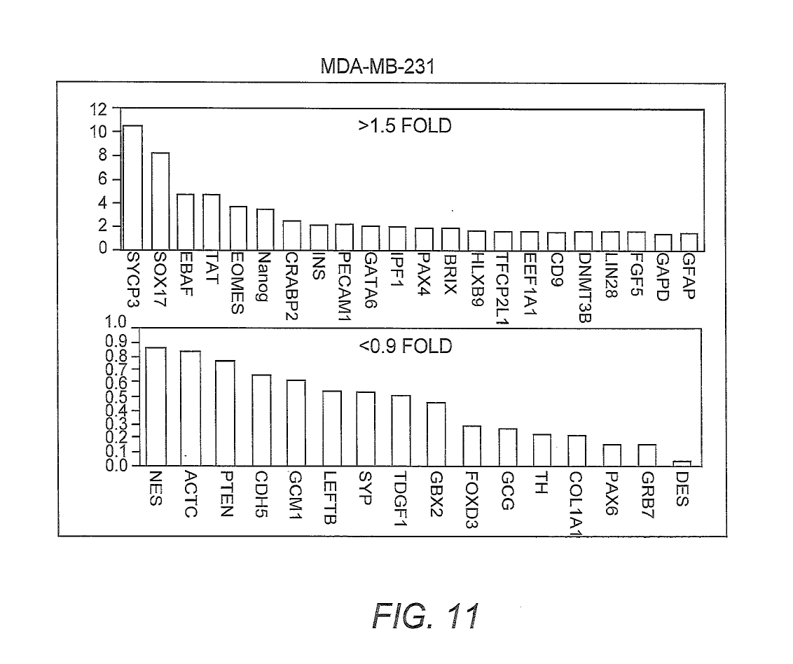

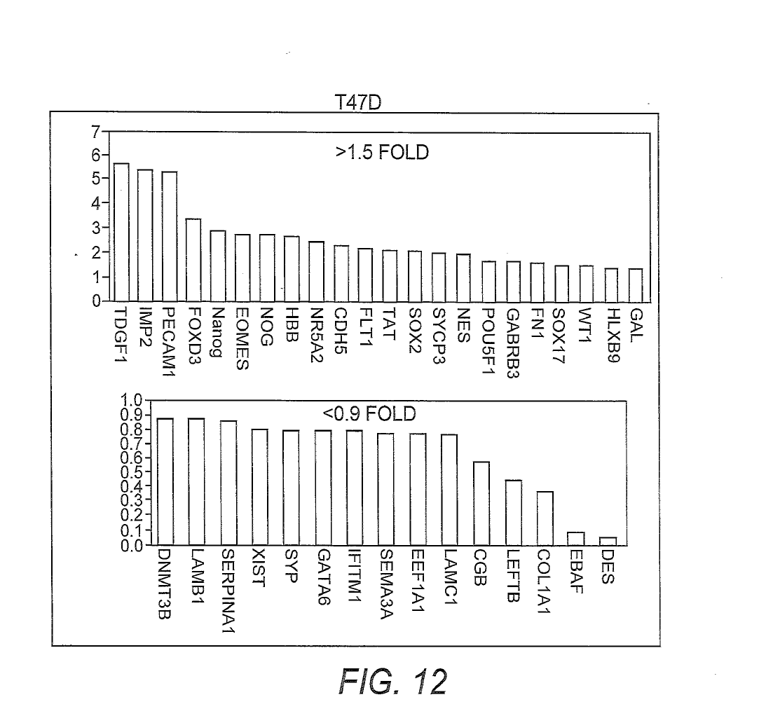

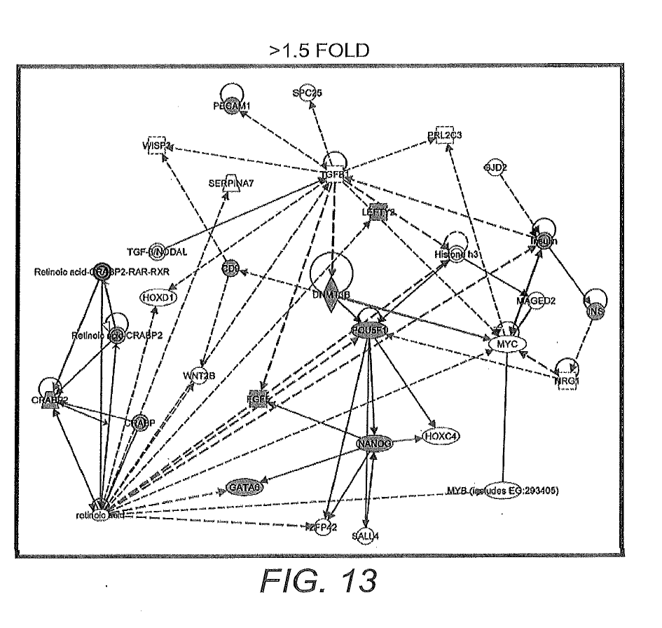

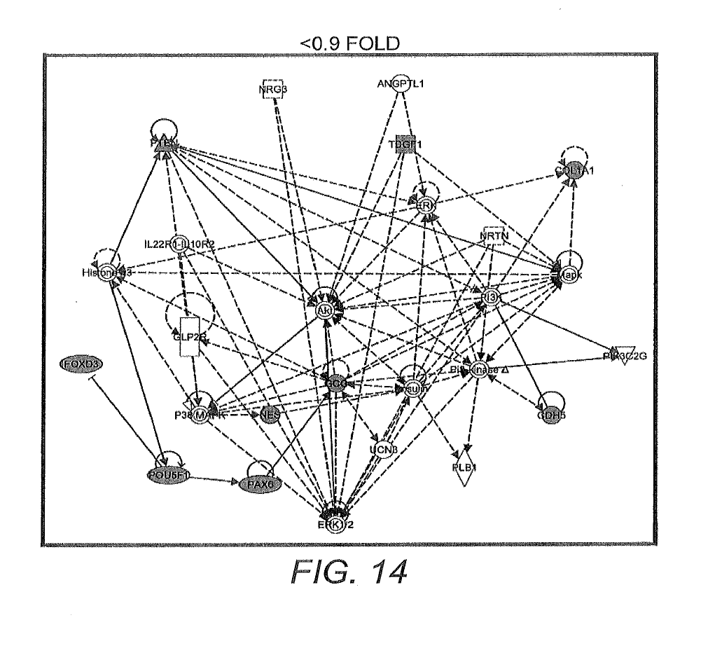

Next, stem cell gene expression was studied in the BCC subsets. Freshly sorted Oct4^{hi} cells were cultured for one week to obtain Oct4^{hi} and Oct4^{med} cells and then compared to the parental post-sorted Oct4⁻ cells. The analyses of stem cell PCR arrays are presented as a fold change of Oct4^{hi/med} vs. Oct4⁻ cells (FIGS. 11 and 1). Although the two different cell types did not show identical changes, the overall functions were comparable, as indicated by Ingenuity Pathway Analyses. The network for genes showing greater than 1.5-fold increases were directly linked to pluripotent markers, such as Nanog, and genes that maintain cell cycle quiescence, such as TGF-β (FIG. 13. The network with genes showing less than a 0.9-fold decrease included those involved in cell proliferation, such as PI(3)K, Akt and p38/MAPK (FIG. 14). Overall, the networks produced indicated increases in genes linked to rapid proliferation and protection from apoptosis for Oct4⁻ BCCs. In contrast, the Oct4^{hi/med} cells mostly expressed genes linked with cell cycle quiescence.

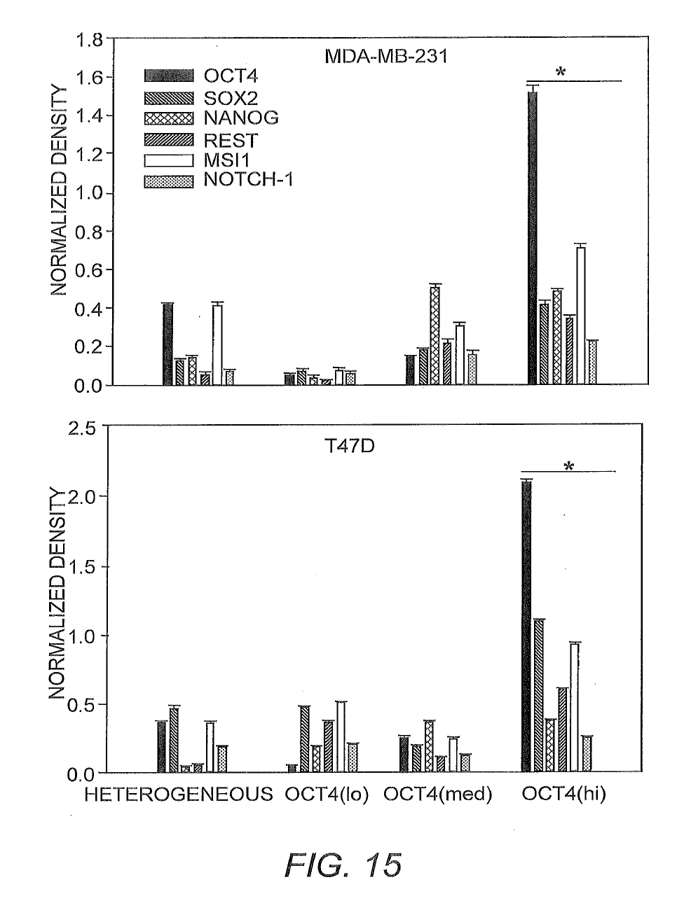

The qPCR array data was validated by western blots for specific stem cell genes with nuclear extracts from Oct4^{hi}, Oct4^{med} and Oct4⁻ MDA-MB-231 and T47D. The stem cell markers employed included Sox2, Musashi-1, Nanog, Notch and REST (Rezza, A. et al. 1010, J. Cell Sci. 123:3256-3265; Singh, S. K. et al. 2008. Nature 453:223-227). For extract purity and normalization purposes, acetylated histone H3 validated nuclear proteins and ribosomal protein L28 identified cytoplasmic proteins. As expected, the intensities of Oct4 bands were denser for Oct4^{hi} than Oct4^{med} and undetectable in the Oct4⁻ subsets (FIG. 15). Although bands were detected for Sox2 and Musashi-1 with Oct4⁻ extracts, the densities were significantly reduced as compared to Oct4^{hi} and Oct4^{med} (FIG. 15). In contrast, Nanog and REST were nearly undetectable in Oct4⁻ but detectable in Oct4^{hi} and Oct4^{med} (FIG. 15). Although Notch was detectable in the unsorted and sorted extracts, the band density was higher in Oct4^{hi} (FIG. 15). Overall, the differences in stem cell gene expression were comparable for both MDA-MB-231 and T47D. However, the variations were more distinct for MDA-MB-231. Similar results with unsorted cells and Oct4⁻ subset were expected due to the low frequency of (Date cells within the unsorted BCCs.

Next, it was determined if there were differences in the expression of hormone receptors (estrogen receptor or ER and progesterone receptor or PR) in Oct4^{hi}, Oct4^{med} and Oct4⁻ BCCs. Intracellular flow cytometry indicated no change in the subsets from triple negative MDA-MB-231. Although the parental T47D were ER⁺/PR⁺, the spread in fluorescence intensities for the three subsets indicated varied receptor levels. The results also indicated a small population that was ER within the Oct4^{hi} subset.

Oct4^{hi} BCCs, therefore, have been shown to have functions consistent with stem cells. Further experiments were performed to determine if Oct4^{hi} BCCs could be serially passed in female nude BALB/c mice. Oct4^{hi}, Oct4^{med} and Oct4⁻ BCCs (200 cells) were injected subcutaneously in the dorsal flanks of mice (P1). In ten mice studied, nine mice formed palpable tumors. At a tumor size of approximately 0.5 cm³, similar cells were acquired from dissociated tumors and the injections were repeated as for P1, until passage 5 (P5). The time for tumor formation in each passage was similar to P1. The tumors in 10 mice injected with Oct4⁻ and Oct4^{med} BCCs regressed after 2 weeks, providing further support for a stem cell role for Oct4^{hi}-expressing cells.

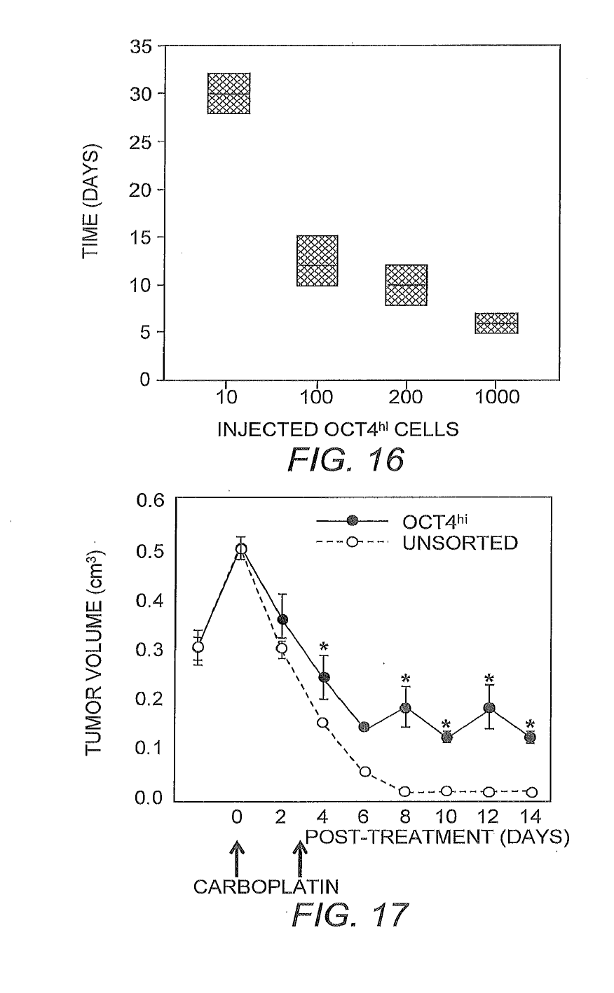

It was then determined if tumors could be initiated with less than 200 Oct4^{hi} BCCs and, if so, whether the formation of palpable tumors was time-dependent. Mice were injected with 10, 100, 200, or 1000 Oct4^{hi} BCCs in the dorsal flank (n=5 per dose group). The time required to attain a tumor size of 0.5 cm³ was plotted against the number of cells injected (FIG. 16). The results indicated that the time required for tumor formation was an inverse function of cell number. Yet, as few as 10 Oct4^{hi} cells were able to form tumors.

Both triple-negative MDA-MB-231 and triple-positive T47D are expected to respond to carboplatin treatment, a drug which acts through a mechanism independent of hormone receptor status. Thus, the responsiveness to carboplatin was investigated in Oct4^{hi} cells from both cells lines. Tumors were injected into the dorsal flanks of mice as before. When the tumors were 0.5 cm³ the mice were injected with carboplatin intra-peritoneally (i.p.). After the second injection of carboplatin, a significant (p<0.05) reduction was observed in the tumors of unsorted BCCs as compared to Oct4^{hi} and also to unsorted BCC not treated with carboplatin. Eight days after treatment with carboplatin, tumors were undetectable in mice injected with unsorted BCCs (FIG. 6C). In contrast, similar treatment with Oct4^{hi} BCCs at the same tumor size as the unsorted BCCs retained palpable tumors (FIG. 17).

It was then determined if Oct4^{hi} BCCs, at sites of distant metastasis, such as bone marrow, could resist carboplatin treatment. Bone marrow was selected for study because it is well-established that BCCs can attain dormancy in this organ, close to the endosteum (Lim, P. K. et al. 2011. Cancer Res. 71:1550-1560; Rao, G. et al. 2004. Cancer Res. 64:2874-2881). Mice were injected intravenously with 10³ Oct4^{hi} MDA-MB-231. After 1 day, carboplatin was injected i.p. at 3-day intervals. One week after the second injection, femurs were decalcified, processed, and examined for GFP⁺ cells. The results indicated there was drug resistance in GFP⁺ BCCs close to the endosteum of bone marrow. Similar cells were not detected in the cellular regions of the marrow, which were flushed from femurs prior to the analyses of the endosteal regions.

It has been reported that BCC quiescence could be partly explained by GJIC between BCCs and bone marrow stroma, which is located close to the endosteum (Lim, P. K. et al. 2011. Cancer Res. 71:1550-1560). As already discussed, Oct4^{hi} BCCs could not regress completely despite treatment with carboplatin. Thus, it was determined if Oct4^{hi} BCCs show preference for GJIC with stroma.

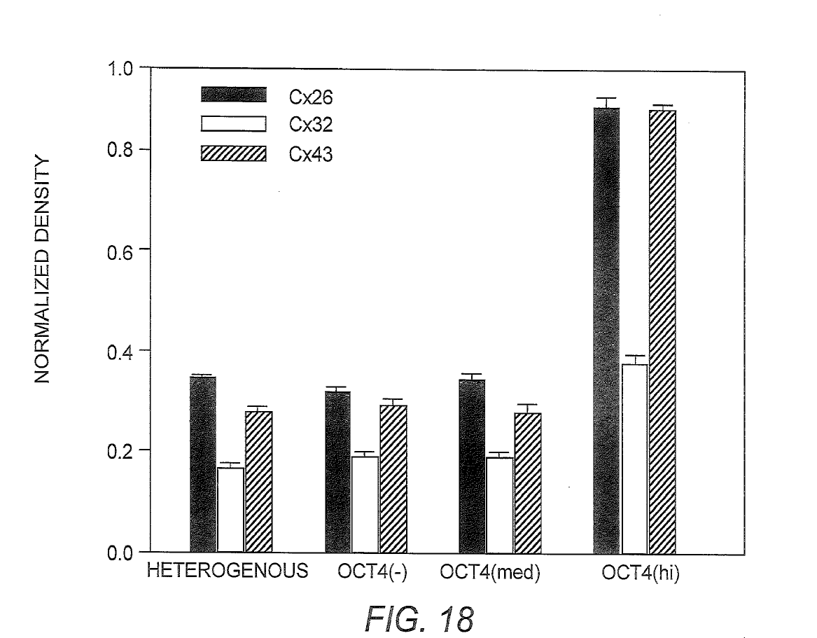

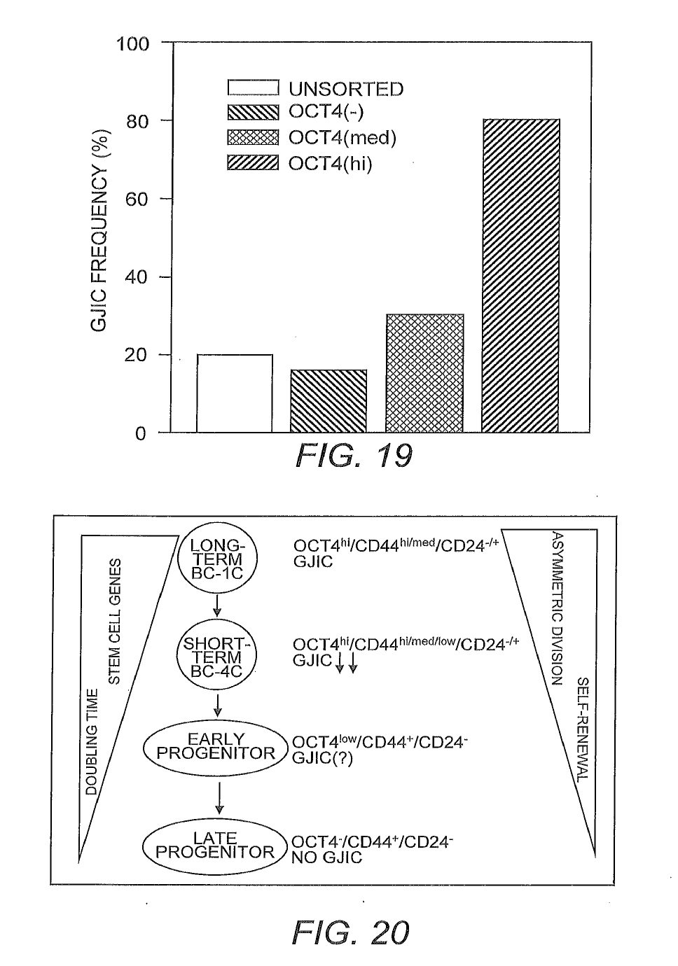

Connexin (Cx) expression was studied using plasma membrane extracts from unsorted, Oct4^{hi}, Oct4^{med} and Oct4⁻ BCCs. Western blots indicated that Cx26, Cx32 and Cx43 were expressed at higher levels in Oct4^{hi} cells, and that Cx43 and Cx26 were expressed at higher levels than Cx32 within the Oct4^{hi} fraction (FIG. 18). It was next determined if the higher expression of connexins in BCCs could provide the cells with an advantage to form GJIC, in co-cultures of Oct4^{hi} BCCs and BM stroma. Immunofluorescence for Cx43 showed intense labeling. As expected, in the presence of the GJIC inhibitor 1-octanol, the intensity of Cx43 was reduced. GJIC was studied via dye transfer between BCCs and stroma using CFDA-SE-labeled Oct4^{hi} BCCs, as described previously (Lim, P. K. et al. 2011. Cancer Res. 71:1550-1560). Dye transfer into stroma was blunted by 1-octanol. Finally, the total number of BCCs showing dye transfer was counted in order to quantify functional GJIC. The results, presented as frequency of GJIC, indicated a significant (p<0.05) increase in GJIC among sorted Oct4^{hi} cells as compared to the other subsets, and Oct4^{med} cells showed significantly (p<0.05) more GJIC than Oct4⁻ and unsorted BCCs (FIG. 19).

Dormancy of the BCC subsets was then studied by first assessing the in vitro invasion abilities of different subsets from MDA-MB-231 and T47D cell lines. Cells, labeled with CFDA-SE, were seeded onto matrigel inserts and then allowed to migrate for 48 hours. Cell migration was based on combining the fluorescence intensities within the lower chambers and underside of the inserts. A significant (p<0.05) increase in invasion by Oct4^{hi} cells as compared to the other subsets was observed.

Since the in vitro studies indicated preference for GJIC with bone marrow stroma (FIG. 19), it was determined if this result could be observed in vivo. Mice were injected intravenously with 10³ unsorted MDA-MB-231 with pEGFP1-Oct3/4. After 72 hours, femurs were flushed to eliminate the cells in the central/cellular areas of the tissue. After this, the femurs were transected longitudinally and then washed to remove loosely adherent cells. The cells contacting the endosteum were gently scraped for the identification of Oct4 expressing cells. The scraped cells were labeled with PE-anti-cytokeratin as a marker for BCCs. Therefore Oct4 expressing cells were detected as yellow cells (green & red) in merged images. Oct4⁻ BCCs did not show GFP emission and therefore remained red. The majority of the cytokeratin⁺ cells were Oct4^{bright}, confirming that this subset contacts the endosteum.

Experiments were performed to determine whether GJIC occurred in vivo by repeating the injections with CFDA-SE-labeled Oct4^{hi} and Oct4⁻ BCCs. The goal was to determine if the BCCs entering bone marrow at the endosteal area can form GJIC, which would be indicated by dye transfer to neighboring cells. Dye transfer was assessed by labeling the scraped cells with PE-anti-cytokeratin. Oct4⁻ cells were mostly yellow due to merging of CFDA-SE dye (green) and PE-anti-cytokeratin (red). In the case of Oct4^{hi} cells, the CFDA-SE dye was observed away from the yellow cells, indicating that CFDA-SE is moving to other cells.

CD44⁺/CD24^{-/low}/lin⁻ have previously been identified as markers of breast cancer stem cells (Al-Hajj, M. et al. 2003. PNAS USA 100:3983-3988). In the present invention, Oct4^{hi} BCCs exhibit tumor-initiating properties. As a result, it was determined if Oct4^{hi} BCCs showed a phenotype consistent with the role of breast cancer stem cells. Two week cultured Oct4^{hi} cells were selected in order to compare the progenies, based on GFP intensities. The cells were studied by flow cytometry for CD44 and CD24 expression. The results indicated that the population with brightest GFP emission included a subset that expressed CD24. The bright GFP cells were dissected by analyzing the upper 5% and it was observed that this subset comprised the CD24⁺ cells. Cells with reduced GFP intensity were mostly negative for CD24, indicating that CD24 expression correlated with Oct4.

Experiments were then performed to determine whether there were differences in CD44 expression. CD24⁺ cells were depleted by negative selection and the remaining cells were analyzed for CD44 by flow cytometry. The results indicated variations in CD44 expression within the Oct4^{hi} subset. Similarly, Oct4^{med} BCCs showed varied CD44, including a subset with dim CD44 expression.

A subset of GFP^{hi} BCCs expressed CD24. Thus, it was determined if this could be related to cell size. Flow cytometry was repeated by gating on the Oct4^{med/hi} population and the results indicated approximately 4% of the cells were CD24⁺. The same subset was analyzed based on forward scatter. The data indicated CD24⁺ cells within the larger subset. A hierarchy of BCCs was constructed, based on phenotype, and is shown in FIG. 20. This hierarchy includes the following designations: Oct4^{hi}/CD44^{hi}/CD24⁺; Oct4^{hi}/CD44^{hi}/CD24⁻; Oct4^{med}/CD44^{hi}/CD24⁻; Oct4^{med}/CD44⁻/CD24⁻; Oct4⁻/CD44^{hi}/CD24⁻. As can be seen from the hierarchy, Oct4^{hi} expression, which has been linked to a variety of stem cell functions that appear related to dormancy in distant tissues such as bone marrow, was not dependent only on the presence of CD44/CD24 expression patterns that had previously been reported, i.e., CD44⁺/CD24⁻ (Ericksson, M. et al. 2007. Mol. Ther. 15:2088-2093).

The results considered together indicate that a subset of Oct4^{hi} BCCs represents a population of cells that can act as cancer stem cells in distant tissues, such as bone marrow. The results that support this conclusion include the identification of these cells in the blood of patients that had undergone chemotherapy or that had negative lymph nodes in the presence of a tumor, and the fact that only high-expressing Oct4 BCCs could sustain tumors in vivo in mice. The finding of Oct4^{hi} cells in a patient that had undergone six cycles of chemotherapy and radiation indicates that Oct4hi BCCs are resistant to treatment, consistent with the resistance to carboplatin demonstrated in mice. Moreover, the result showing negative lymph nodes with the presence of Oct4^{hi} BCCs in blood lends support to the debate concerning the lack of utility of lymph node dissection in some breast cancer treatment (Guiliano, A. E. et al. 2011. JAMA 305:569-575; Kawada, K. and M. M. Taketo. 2011. J. Clin. Oncol. 26:2813-2820). Thus, the BCC subset identified in the present invention may be a useful marker for breast cancer diagnosis and prognosis that has greater utility than lymph node dissection alone.

Another important finding of the present invention was the fact that the BCC subset of Oct4^{hi} cells may be a more primitive cell group than stem cells. This conclusion was reached based on the finding that stem-like subpopulations within tumors cannot be clonally expanded sue to spontaneous differentiation, yet, while maintaining the Oct4^{hi} BCCs in culture, the subset was able to be expanded since the cells differentiated into Oct4med and Oct4low cells. Thus, the Oct4^{hi} BCC subset, exhibits stem cell properties but also adapts dormancy in distant sites, leading to evasion of treatment. Given that a large percentage of cancer resurgence has been purported to occur in cells from bone marrow (Pantel, K. and M. Otto. 2001. Sem. Cancer Biol. 11:327-337), the finding of a marker for a population of such BCCs will provide for new insights into cancer therapy, cancer diagnosis and cancer prognosis.

The ability of Oct4^{hi} cells to enter bone marrow and resist chemotherapy is an important finding for investigation into the effects of the microenvironment on cancer cell survival. It is possible that immune mediators in the microenvironment serve to protect Oct4^{hi} cells and opens new areas for investigation. The finding that Oct4^{hi} cells were dependent on stem cell networks while Oct4^{low} cells exhibited proliferation-associated genes may explain a mechanism where the Oct4^{hi} cells maintain dormancy in bone marrow.

Finally, although CD44/CD24 expression has been a accepted method for isolating BCC stem cells, the results of the present investigations indicate that the cellular properties of Oct4 expression are independent of CD44/CD24 status. In fact, Oct4 has now been identified as a unique marker for identifying stem-like subsets of cells in breast tumors. Moreover, the results of the present invention indicate that establishing a single phenotype for tumor-initiating cells is not likely. Instead, a hierarchy of such cells has now been identified, a hierarchy based on function and phenotype. The hierarchy now discovered combines the Oct4, CD44 and CD24 status of different breast cancer cell subsets, with Oct4^{hi} cells identified as the most primitive cell type. Thus, the present invention is a population of BCCs that have stem cell characteristics but also are identified as exhibiting functional GJIC with bone marrow stroma, indicating the cells establish dormancy and would remain resistant to chemotherapy. Thus, these cells provide a useful target for testing new cancer therapies for their ability to affect cancer dormancy and reverse drug resistance.

Contemplated by the present invention is a biomarker for breast cancer based on the detection of Oct4^{hi} cells within samples taken from a patient, such as tissues or biological fluids of patients. Commonly used patient samples would include but not be limited to tumor samples, blood samples, or bone marrow samples. These patient samples can be screened for the presence of Oct4 cells of various types, i.e., Oct4^{hi}, Oct4^{med}, Oct4^{low}. The cells identified may also be screened for CD44 and CD24 status as a further method for phenotyping cells as breast cancer stem cells. In a preferred embodiment, the breast cancer biomarker of the present invention comprises a breast cancer cell with a phenotype Oct4^{hi}/CD44^{hi/med}/CD24^{-/+}. In the present invention it has been shown that cells with the Oct4^{hi}/CD44^{hi/med}/CD24^{-/+} phenotype represent dormant, metastatic breast cancer cells that can exist in bone marrow.

The Oct4 expression profile and Oct4 subset analysis also provides for a method of cancer prognosis and diagnosis. Cancer prognosis would be aided by identifying whether the patient has Oct4⁻ cells that are likely dormant cells which are drug resistant. The presence of the Oct4^{hi} cells would indicate that a patient's cancer would, therefore, be identified as one that is likely to reoccur, indicating a poor prognosis. As a result, the present invention is also a method for developing a breast cancer prognosis for a patient which comprises detecting the presence of the biomarker cells of the present invention in a patient sample wherein the presence of said biomarker is indicative of a the presence of dormant metastatic breast cancer cells in the patient and a poor prognosis for the patient. In the context of the present invention a “poor” prognosis is one where cancer reoccurrence is expected to occur. Additionally, the present invention is a method of diagnosing metastatic breast cancer which comprises detecting the presence of the biomarker of the present invention in a patient sample, wherein the presence of the biomarker indicates that the patient has metastatic breast cancer. Again, the patient samples would include but not be limited to tumor samples, blood, or bone marrow samples.

Finally, the Oct4^{hi} cells themselves provide for a useful system for testing new therapies for their ability to affect dormant cancer cells, which could lead to new treatments for cancer. Thus, another object of the present invention is a method for identifying chemotherapeutic agents that can kill dormant metastatic breast cancer cells in bone marrow which comprises that steps of contacting a breast cancer cell with a phenotype Oct4^{hi}/CD44^{hi/med}/CD24^{-/+} in vitro with an agent; and determining whether the agent is capable of killing said breast cancer cell, wherein death of said breast cancer cell is indicative of the ability of the agent to kill dormant metastatic breast cancer cells in bone marrow.

Although specific embodiments of the present invention have been described, it should be understood that such embodiments are by way of example only and merely illustrative of but a small number of the many possible specific embodiments that can represent applications of the principles of the present invention. Various changes and modifications obvious to one skilled in the art to which the present invention pertains are deemed to be within the spirit, scope and contemplation of the present invention as further defined in the appended claims.

The following examples are provided to further illustrate the present invention.

### EXAMPLES

### Example 1

### Reagents and Antibodies

Liquid and 10× powdered DMEM were purchased from Gibco (Grand Island, N.Y.). Noble agar, propidium iodide, fetal bovine sera, RPMI 1640, Hoechst 33342 dye, verapamil, and mouse monoclonal IgG to β-actin were purchased from Sigma (St. Louis, Mo.). Biocoat Matrigel Matrix, anti-human CD44-APC, and anti-human CD24-PE were purchased from BD Biosciences (Franklin Lakes, N.J.). Vybrant CFDA-SE Cell Tracer, CyQUANT Cell Proliferation Assay, Platinum SYBR Green qPCR SuperMix-UDG Kit, SuperScript III reverse transcriptase, RNase A, Platinum Taq polymerase, Dynabeads pan mouse-IgG, Connexin Antibody Sampler Pack, and Geneticin G418 were purchased from Invitrogen (Carlsbad, Calif.). HyGLO HRP Chemiluminescent Detection Kit was purchased from Denville Scientific (Metuchen, N.J.). Restore Western Blot Stripping Buffer and NE-PER Nuclear and Cytoplasmic Extraction Kit were purchased from Thermo Scientific (Waltham, Mass.).

The following antibodies were purchased from Abcam (Cambridge, Mass.): rabbit polyclonal anti-Oct4, mouse anti-progesterone receptor (PR) mAb, rabbit polyclonal anti-estrogen receptor (ER) a, rabbit polyclonal anti-Sox2, rabbit polyclonal anti-Nanog, rabbit polyclonal anti-Musashi, rabbit polyclonal anti-ABCG2, rabbit polyclonal anti-REST and FITC-polyclonal goat anti-rabbit IgG. APC-anti-rabbit IgG and polyclonal goat anti-ribosomal protein L28 were purchased from Santa Cruz Biotechnology (Santa Cruz, Calif.). Rabbit polyclonal IgG to acetyl-histone H3 was purchased from Upstate Cell Signaling Solutions (Lake Placid, N.Y.). Antibodies to p15, p16, Cdk4, and cyclin D1 were purchased from Cell Signaling Technology.

### Example 2

### Human Subjects

The use of all human tissues was approved by the Institutional Review Board of the University of Medicine and Dentistry of New Jersey-Newark Campus. All subjects signed the approved consent forms. Stromal cells were cultured from bone marrow aspirates. Left over surgical tissues from malignant (invasive ductal carcinoma) and normal areas of the mammary gland was obtained from Brookdale University Hospital, Brooklyn, N.Y. The studies were approved by the Institutional Review Board of UMDNJ and Brookdale University Hospital. Peripheral blood was obtained from patients with breast cancer.

### Example 3

### Isolation of BCC Subsets

MDA-MB-231 and T47D were stably transfected with pEGFP1-Oct3/4. This vector expresses green fluorescent protein (GFP) under the control of Oct4 regulatory region (Gerrard, L. et al. 2005. Stem Cells 23:124-133). Dose response toxicity curves indicated that 600 μg/ml G418 as optimum for MDA-MB-231 and 400 μg/mL for T47D. The stable transfectants were maintained in the same concentration of G418. Immediately before all assays, the cells were sorted with the FACSDiva (BD Biosciences). Selection of subsets was based on the intensity of GFP of singlets. The top 5% was designated Oct4^{hi} and the lower 5%, Oct4⁻. Those between the two extremes were designated Oct4^{med}. GFP intensity correlated with Oct4 protein, as indicated by immunofluorescence for intracellular Oct4.

### Example 4

### Gap Junctional Intercellular Communication (GJIC)

GJIC was assessed by CFDA-SE dye exchange from BCCs to stroma in co-cultures, as described previously (Ramkisson, S. H. et al. 2007. Cancer Res. 67:1653-1659). Briefly, BCCs and stroma were co-cultured at equal ratios in α-MEM with 10% FCS, in the presence or absence of 300 μM 1-octanol. CFDA-SE dye transfer was assessed on an EVOS fl fluorescence imager (AMG Micro, Bothell, Wash.).

### Example 5

### Real-Time PCR

RNA extraction was performed via RNeasy Mini Kit from (Qiagen, Valencia, Calif.). Total RNA (1 μg) were immediately reverse transcribed using dNTPs (0.2 mM), random hexamers (50 μM), and SuperScript III reverse transcriptase (200 U). Incubation conditions were 25° C. for 5 min, 50° C. for 60 min, and 70° C. for 15 min. Real-time PCR was performed with 200 ng cDNA using Platinum SYBR Green qPCR SuperMix-UDG Kit (Invitrogen) and then analyzed on the 7300 Real-Time PCR System (Applied Biosystems, Foster City, Calif.). The analyses were performed with an initial incubation of 50° C. for 2 min followed by 95° C. for 2 min. After this, the cycling conditions were as follows: 94° C. for 15 sec and 60° C. for 45 sec, for 40 cycles. Primer sequences are described in Table S3.

### Example 6

### Array Analyses

Gene expression analyses were performed with Taqman Stem Cell Pluripotency Array (Applied Biosystem), by quantitative RT-PCR, using ABI 7900. The fold change between subsets were calculated using the ΔΔCt method as follows: (Ct_{Oct4(hi)}−Ct_{Gene of Reference})/(Ct_{Oct4(-)}−Ct_{Gene of Reference}).

The fold changes were entered into Ingenuity Pathway Analysis (Ingenuity® Systems, www.ingenuity.com) for pathway networks, as described previously (Lim, P. K. et al. 2011. Cancer Res. 71:1550-1560). The analyses allowed for the identification of complex biological interactions based on at least one published reference in the database. Biological predictions were made based on protein-protein interactions, and the insights into molecular pathways were gathered.

### Example 7

### Decalcification and Processing of Murine Femurs

Mice were injected intravenously with 10³ BCCs, stably transfected with pEGFP1-Oct3/4. After 24 hours, mice were injected i.p. with carboplatin (50 mg/kg), followed by a second dose after 3 days. One week after the final injection of carboplatin, mice were euthanized, and the femurs were removed. Femurs were rinsed and the cells flushed using a 27 gauge needle attached to a syringe with PBS to remove the cells within the central region of the cavity. After this, the femurs were fixed overnight in 4% formaldehyde at 4° C. After this, the femurs were transferred to decalcification solution (Cal-Ex Decalcifier, Fisher Scientific, Pittsburgh, Pa.) overnight at 4° C. After this, femurs were rinsed in running distilled water for 4 hours and then embedded in Optimal Cutting Temperature (O.C.T.) compound (Tissue-Tek, Redding, Calif.). After this, tissues were section in 10 μm with a cryostat-microtome HM550 (Walldorf, Germany). Slides were examined with an EVOS fl fluorescence imager.

### Example 8

### Immunocytochemistry

BCCs were added to sterile coverslips placed within 6-well plates. The next day, after adherence, cells were washed with 1×PBS, fixed with 3.7% formaldehyde for min, permeabilized with 0.1% Triton X-100 in PBS and blocked in 1% BSA in PBS for 1 h. The blocking buffer was washed with PBS and the cells were incubated with anti-Oct4 (1:500 dilution). The antibody was diluted in 0.1% BSA/0.1% Triton X-100 in PBS. After 30 min, the cells were washed in PBS and then incubated with goat anti-rabbit IgG-FITC (1:1000 dilution) for 2 h in the dark. Nuclei were stained with 300 nM DAPI, and F-actin was stained with Texas Rex-X phalloidin. Green emission was observed using the 518 nm filter.

### Example 9

### Immunohistochemistry

Oct4 staining in primary breast tissues was performed by fixing in 4% paraformaldehyde overnight followed by incubation in 20% sucrose overnight. The tissues were embedded in O.C.T. compound and then sectioned into 5 μm slices as described above. Sections were placed on slides and then de-paraffinized in xylene. After this, the sections were rehydrated with consecutive washes in decreasing concentrations of ethanol: 100%, 90%; 80% 70%. Slides were washed twice in PBS and then incubated in 0.25% Triton X-100 for 5 min. This followed by blocking in 1% BSA for 1 h. Slides were incubated overnight at 4° C. with anti-Oct4 (1:500 dilution). The antibody was diluted in PBS containing 0.1% Triton X-100 and 0.1% BSA. Diaminobenzidine (DAB) detection for Oct4 was performed using the DAKO Envision+System-HRP according to manufacturer's protocol.

### Example 10

### Extract Preparation/Western Blotting

Western blots were performed as previously described (Trzaska, K. A. et al. 2007. Stem Cells 25:2797-2808). Cell extracts from surgical tissues were obtained by homogenizing in the NP-40 cell lysis buffer containing protease inhibitors (Invitrogen). For intracellular proteins with cell lines, whole cell extracts were prepared with the NP-40 buffer and also nuclear/cytoplasmic extracts with NE-PER Nuclear and Cytoplasmic Extraction kit. For membrane proteins, extracts were prepared with Qproteome Plasma Membrane Protein kit (Qiagen).

BCC extracts (20 μg) were subjected to electrophoresis on 4-20% SDS-PAGE (Bio-Rad; Hercules, Calif.). Proteins were transferred to PVDF membranes, and membranes were incubated overnight in the respective primary antibodies. This was followed by 2 h incubation with HRP-conjugated secondary antibodies at 1:2000 final dilutions. The latter was detected with chemiluminescence. Membranes were stripped with Restore Western Blot Stripping Buffer and then re-probed for other proteins, including β-actin mAb (1:4000 dilution). All bands were normalized to (3-actin.

### Example 11

### Tumorsphere Assay and In Vitro Serial Passage

BCC subsets were seeded at one cell per well in serum-free media in 96-well low-adhesion plates (Costar, Corning, N.Y.). At day 10, wells with spheres containing greater than 20 cells were designated as tumorsphere-positive. One tumorsphere was dissociated, first enzymatically with trypsin and then mechanically with a syringe attached to a 27-gauge needle. After this, the cell suspension was passed through a 40 μm mesh (BD cell strainer cap tube). One cell, with similar phenotype, was re-assessed for green fluorescence and then re-seeded at 1 cell/well. This method was continued serially more than 5 times.

### Example 12

### Noble Agar Assay

The assay was established with two layers of noble agar in 60 mm Petri dishes. The bottom layer contained 4 mL of 0.6% agar, and the top layer contained the cells in 4 mL of 0.3% agar. The agar was prepared with a stock of 1.8% diluted in deionized water. Agar was autoclaved and then diluted to the working concentration with sterile deionized water and 2×DMEM. The bottom agar was allowed to solidify at 37° C. for 10 min. After this, the top agar was added with BCCs at concentrations between 10¹ and 10⁵ at log₁₀ dilutions. Plates were incubated and examined with EVOS fl fluorescence imager.

### Example 13

### Flow Cytometry

Intracellular flow cytometry for Oct4 was performed by the following consecutive treatments: fixed in 4% formaldehyde for 15 min at 4° C., permeabilized in 0.1% Triton X-100 for 30 min, incubated with anti-Oct4 for 30 min at 4° C., washed once with cold PBS and then incubated with goat anti-rabbit IgG-APC for 30 min in the dark at 4° C. After this, cells were washed with PBS and then immediately analyzed on the FACSCalibur (BD Biosciences).

Cell surface labeling for CD44/CD24 with pEGFP1-Oct3/4 stable transfectants were performed by first washing with PBS, fixing in 4% formaldehyde as for intracellular labeling, incubating with anti-CD44-APC for 30 min followed by a second labeling with anti-CD24-PE. All incubations occurred for 30 min at 4° C. in 2% FBS/PBS. The cells were immediately analyzed by gating cells, based on green (GFP) emission, with the FACSCalibur. The data were analyzed with CellQuest software (BD Biosciences).

Side population analysis of the pEGFP1-Oct4 stable transfectants was performed using the LSR II (BD Biosciences). Stable transfectants (10⁶) were washed in PBS, resuspended in phenol-free, Ca²⁺/Mg²⁺-free 1× Hank's Balanced Salt Solution containing 2% FBS and then incubated in titrations of Hoechst 33342 and verapamil. Optimal titrations and conditions were determined to be 5 μg/ml Hoechst 33342 (90 min incubation at 37° C.) and 400 μM verapamil (10 min pre-incubation). Cells were then washed, maintained on ice, and incubated in propidium iodide (5 μg/ml) to gate for viability. The analyses were done by gating on the top and lower 5% of GFP-expressing cells, designated Oct4^{hi} and Oct4⁻, respectively. The cells between the two extremes were also analyzed (Oct4^{med}).

### Example 14

### Doubling Time

Doubling time was performed by seeding BCCs at 5×10³/well into 96-well plates. After four days cell numbers were determined by CyQUANT Cell Proliferation Assay Kit (Invitrogen). Cell numbers were calculated on a standard curve of fluorescence intensity vs. known cell densities. Calculations for doubling times were based on the following: A=A₀*2ⁿ, where A=final cell number, A₀=initial seeding density and n=number of divisions. Doubling time was taken by dividing the incubation time by the number of divisions. The fluorescence method was validated by manual cell count.

### Example 15

### Cell Cycle Analyses

Cell cycle analyses were performed with BCCs (10⁶). Cells were washed in PBS and then resuspended in 0.1% hypotonic sodium citrate solution containing 5 μg/ml propidium iodide and 200 μg/ml DNase-free RNase A. Cells were incubated for 30 min at room temperature and then immediately analyzed on FACSCalibur (BD, San Jose, Calif.).

### Example 16

### Invasion Assay

BD BioCoat™ Matrigel™ Matrix (0.2 ml) was added into 8 μm FluoroBlok cell culture inserts. These inserts prevent the plate reader from detecting emission in the upper chamber. After solidification of the matrigel at 37° C. for 1 h, the inserts were placed in 24-well culture plates containing 0.5 mL DMEM with 10% FCS. BCCs (2×10⁴) in sera-free media were added to the inner wells. The cells were allowed to migrate for 2 h at 37° C. After this, the inserts were removed, and the cells within the inner chambers were gently removed with a Q-tip. The wells under the membranes were then transferred to another well for labeling with 10 μM CDFA-SE, diluted in PBS for 1 h. After this, the wells were gently washed with PBS to remove excess CFDA-SE and then transferred to another well containing PBS for analyses on Victor 3V Multi-well plate reader (Perkin Elmer, Waltham, Mass.) at 485 nm/535 nm. Controls included MCF12A non-tumorigenic breast epithelial cells.

### Example 17

### Time-lapse Microscopy

Time-lapse microscopy of MDA-MB-231 cells was performed with Axiovert 200M fluorescence microscope (Carl Zeiss, Inc.) at constant conditions of 37° C. and 5% CO₂. Brightfield and fluorescence images were acquired every 10 min for up to 68 h using a 10× objective (Zeiss) and an AxioCam MRm camera with Axiovision software v4.6 (Zeiss). Individual images were adjusted for brightness using the Axiovision software and exported to ImageJ (National Institutes of Health, Bethesda, Md.), where the movies were assembled. Individual cells were tracked manually.

### Example 18

### In Vivo Serial Passages and Carboplatin Treatment

Female athymic BALB/c mice (4 weeks) were obtained from Harlan Laboratories (Somerville, N.J.) and housed in a laminar flow hood at an AAALAC-accredited facility. The use of mice was approved by the Institutional Animal Care and Use Committee, New Jersey Medical School (Newark, N.J.).

Serial passage of different BCC subsets was performed by injecting BCCs in dorsal flanks of mice. The cells were resuspended in PBS and then mixed with matrigel at 1:1 ratio in 0.2 mL total volume. The cells were injected at different numbers, but in a constant volume to study dose effects on the time for tumor growth. Parallel analyses were performed with unsorted BCCs. Tumors were monitored daily for 30 days and measured in two dimensions with a caliper and volume was calculated using the following: formula V=πr²h, where r=radius and h=height. Subsequent passages occurred by sorting the same cell subset (top 5% highest GFP) and then repeating the injection as above.

Migration of BCC subsets to bone marrow of nude mice was studied by intravenous injection of 10³ pEGFP1-Oct3/4 stable transfectants. After 72 h, the femur was flushed to eliminate the cells within the central region. After this, femurs were transected longitudinally and the endosteal cells were acquired by scraping with a blunt spatula. Cells were then labeled by immunocytochemistry for cytokeratin, as described above.

GJIC was assessed by dye transfer from BCCs to stroma in nude BALB/c was investigated by injecting CFDA-SE-labeled Oct4^{hi} BCCs intravenously. After 72 h, the endosteal region cells were collected as above and then labeled for cytokeratin.

Carboplatin responses to tumor growth were assessed by subcutaneous injection of 200 Oct4^{hi} BCCs in matrigel in the dorsal flank. For unsorted BCCs, 10⁶ cells were injected. At −0.5 cm³ tumors, mice were injected with carboplatin (50 mg/kg), twice, at 3-day intervals. Tumor sizes were recorded every 2 days with a caliper, as described for serial passage.

### Example 19

### Culture of Human Mesenchymal Stem Cells (MSCs)

Extracts from MSCs served as positive control in the western blots for Oct4B. MSCs were cultured from bone marrow (BM) aspirates as described (Greco, S. J. et al. 2007. Stem Cells 25:3143-3154). The use of human bone marrow aspirates followed a protocol approved by the Institutional Review Board of The University of Medicine and Dentistry of New Jersey-Newark campus. Unfractionated bone marrow aspirates were cultured in DMEM with 10% FCS in Falcon 3003 dishes. After 3 days, red blood cells and granulocytes were removed with Ficoll Hypaque. After four cell passages, the adherent cells were asymmetric, CD14⁻, CD29⁺, CD44⁺, CD34⁻, CD45⁻, SH2⁺, prolyl-4-hydroxylase⁻ (Potian et al., 2003).

### Example 20

### Chemical Induction of Green Fluorescence Protein (GFP) in Oct4(−) Breast Cancer Cells (BCCs)

At 40-50% confluence stably transfected BCCs were treated with G9a histone methyltransferase inhibitor, [2-(Hexahydro-4-methyl-1H-1,4-diazepin-1-yl)-6,7-dimethoxy-N-[1-(phenylmethyl)-4-piperidinyl]4-quinazolinamine] (BIX01294, Enzo Life Sciences, Farmingdale, N.Y.). BIX01294 and similar chemicals can induce Oct4 in negative cells, in reprogramming (Huangfu, D. et al. 2008. Nat. Biotech. 26:795-797; Shi, Y. et al. 2008. Cell Stem Cell 3:568-574). Cells were treated with 2.7 μM BIX01294. After 24 h, images were taken by fluorescence microscopy. Control cells were untreated or treated with vehicle (dimethyl sulphoxide, DMSO).

What is claimed is:

1: A breast cancer biomarker comprising a breast cancer cell with a phenotype Oct4^{hi}/CD44^{hi/med}/CD24^{-/+}.

2: The biomarker of claim 1 wherein said breast cancer cell establishes gap junctional intercellular communication with bone marrow stroma.

3: A method for developing a breast cancer prognosis comprising detecting the presence of the biomarker of claim 1 in a patient sample wherein the presence of said biomarker is indicative of a the presence of dormant metastatic breast cancer cells in the patient and a poor prognosis for the patient.

4: A method for diagnosing metastatic breast cancer comprising detecting the presence of the biomarker of claim 1 in a patient sample, wherein the presence of the biomarker of claim 1 indicates that the patient has metastatic breast cancer.

5: A method for identifying chemotherapeutic agents that can kill dormant metastatic breast cancer cells in bone marrow comprising:
a) contacting a breast cancer cell with a phenotype Oct4^{hi}/CD44^{hi/med}/CD24^{-/+} in vitro with an agent; and
b) determining whether the agent is capable of killing said breast cancer cell, wherein death of said breast cancer cell is indicative of the ability of the agent to kill dormant metastatic breast cancer cells in bone marrow.
